(19) United States
(12) Patent Application Publication
(10) Pub. No.: US 2002/0058869 A1
(43) Pub. Date: May 16, 2002

(54) Methods of magnetic resonance imaging (MRI) using contract agent solutions formed from the dissolution of hyperpolarised materials

(76) Inventors: Oskar Axelsson, Malmo (SE); Haukur Johannesson, Malmo (SE); Jan Henrik Ardenkjaer-Larsen, Malmo (SE); Ib Laursen, Lyngby (DK)

Correspondence Address: Amersham Biosciences, 800 Centennial Avenue, Piscataway, NJ, 08855, US

(21) Appl. No.: 09/990,512

(22) Filed: Nov. 16, 2001

Related U.S. Application Data
(63) Continuation of application No. PCT/GB00/01888, filed on May 17, 2000.
(60) Provisional application No. 60/139,258, filed on Jun. 15, 1999.
(60) Provisional application No. 60/146,947, filed on Aug. 2, 1999.

(30) Foreign Application Priority Data
May 19, 1999 (GB) 9911682.4
Jul. 14, 1999 (GB) 9916567.2

(51) Int. Cl. A61B 5/05; G01V 3/00

(52) U.S. Cl. 600/423; 324/321

(57) ABSTRACT

The present invention provides a method of magnetic resonance investigation of a sample, preferably of a human or non-human animal body, said method comprising:

(i) nuclear spin polarizing a solid MR imaging agent (i.e. a material containing in its molecular structure a non-zero nuclear spin nucleus) by (a) spin refrigeration, or by, (b) irradiating with circularly polarized light;

(ii) administering the nuclear spin polarized MR imaging agent to said sample, preferably after dissolution in a physiologically tolerable solvent and also preferably after separation from some or all of the paramagnetic species or chromophores;

(iii) exposing said sample to a radiation at a frequency selected to excite nuclear spin transitions in selected nuclei therein, e.g. the spin polarized nuclei of the MR imaging agent;

(iv) detecting magnetic resonance signals from said sample; and

(v) optionally generating an image, dynamic flow data, diffusion data, perfusion data, physiological data (e.g. pH, pO₂, pCO₂, temperature or ionic concentrations) or metabolic data from said detected signals.

The present invention relates to methods of magnetic resonance imaging (MRI), and in particular to the use therein of contrast agent solutions formed from the dissolution of hyperpolarised materials. In addition, a novel polarisation method of solid materials is disclosed.

Magnetic resonance imaging is a diagnostic technique that has become particularly attractive to physicians as it is non-invasive and does not involve exposing the patient under study to potentially harmful radiation such as X-rays.

In order to achieve effective contrast between MR images of different tissue types, it has long been known to administer to the subject MR contrast agents (e.g. paramagnetic metal species) which affect relaxation times in the zones in which they are administered or at which they congregate. By shortening the relaxation times of the imaging nuclei (the nuclei whose MR signal is used to generate the image) the strength of the MR signal is changed and image contrast is enhanced.

MR signal strength is also dependent on the population difference between the nuclear spin states of the imaging nuclei. This is governed by a Boltzmann distribution and is dependent on temperature and magnetic field strength. However, in MR imaging contrast enhancement has also been achieved by utilising the “Overhauser effect” in which an esr transition in an administered paramagnetic species is coupled to the nuclear spin system of the imaging nuclei.

Techniques have also been developed which involve ex vivo nuclear spin polarisation of agents containing non zero nuclear spin nuclei (e.g. ³He), prior to administration and MR signal measurement. Some such techniques involve the use of polarising agents, for example conventional OMRI contrast agents or hyperpolarised gases to achieve ex vivo nuclear spin polarisation of non zero nuclear spin nuclei in an administrable MR imaging agent. By polarising agent is meant any agent suitable for performing ex vivo polarisation of an MR imaging agent.

The ex vivo method has the advantage that it is possible to avoid administering the whole of, or substantially the whole of, the polarising agent to the sample under investigation, whilst still achieving the desired nuclear spin polarisation in the MR imaging agent. Thus the method is less constrained by physiological factors such as the constraints imposed by the administrability, biodegradability and toxicity of OMRI contrast agents in in vivo techniques.

MRI methods involving ex vivo nuclear spin polarisation may be improved by using nuclear spin polarised MR imaging agents comprising in their molecular structure nuclei capable of emitting MR signals in a uniform magnetic field (e.g. MR imaging nuclei such as ¹³C or ¹⁵N nuclei) and capable of exhibiting a long T₁ relaxation time, and preferably additionally a long T₂ relaxation time. Such agents are referred to hereinafter as “high T₁ agents”. A high T₁ agent, a term which does not include ¹H₂O, will generally be water-soluble and have a T₁ value of at least 6 seconds in D₂O at 37° C. and at a field of 7 T, preferably 8 secs or more, more preferably 10 secs or more, especially preferably 15 secs or more, more especially preferably 30 secs or more, yet more especially preferably 70 secs or more, even yet more especially preferably 100 secs or more. Unless the MR imaging nucleus is the naturally most abundant isotope, the molecules of a high T₁ agent will preferably contain the MR imaging nucleus in an amount greater than its natural isotopic abundance (i.e. the agent will be “enriched” with said nuclei).

The use of hyperpolarised MR contrast agents in MR investigations such as MR imaging has the advantage over conventional MR techniques in that the nuclear polarisation to which the MR signal strength is proportional is essentially independent of the magnetic field strength in the MR apparatus. Currently the highest obtainable field strengths in MR imaging apparatus are about 8 T, while clinical MR imaging apparatus are available with field strengths of about 0.2 to 1.5 T. Since superconducting magnets and complex magnet construction are required for large cavity high field strength magnets, these are expensive. Using a hyperpolarised contrast agent, since the field strength is less critical it is possible to make images at all field strengths from earth field (40-50 μT) up to the highest achievable fields. However there are no particular advantages to using the very high field strengths where noise from the patient begins to dominate over electronic noise (generally at field strengths where the resonance frequency of the imaging nucleus is 1 to 20 MHz) and accordingly the use of hyperpolarised contrast agents opens the possibility of high performance imaging using low cost, low field strength magnets.

It has previously been found (see the present Applicant's earlier International Publication No. WO 99/35508, the enclosures of which are hereby incorporated by reference) that MR imaging agents (e.g. high T₁ agents) may be nuclear spin polarised in the solid state prior to being dissolved in a physiologically tolerable solvent and subsequently administered as a hyper-polarised solution to the sample under investigation. Furthermore, where the polarisation is effected by means of a polarising agent, the whole, substantially the whole, or at least a portion of the polarising agent can be separated from the MR imaging agent prior to administration.

However, there is still a need for efficient methods of ex vivo nuclear spin polarisation of MR imaging agents while in the solid state. It has now been realised that this can be achieved by spin refrigeration or by irradiating with circularly polarised light, as described below.

The spin refrigerator technique or spin refrigeration involves placing the material which is to be spin polarised, doped with or in intimate admixture with the paramagnetic ions, in a strong magnetic field at a low temperature and repeatedly or continuously re-orienting the material relative to the magnetic field, e.g. about an axis perpendicular to the field axis. See for example Jeffries in Proc. Int. Conf. on Polarised Targets and Ion Sources, Saclay, France, 1967, 147 (1966) and McColl et al. Phys Rev B. 7: 2917 (1970) and references therein.

The present invention relates in one aspect to the use of light irradiation to generate nuclear-spin hyperpolarized MR imaging agents by irradiation of a solid compound having a singlet electronic ground state or alternatively generating hyperpolarized MR imaging agents by spin refrigeration. The former is achieved by generation of a polarized triplet electronic state in the solid compound and transformation of the electronic state polarization into a nuclear spin state population difference in a solid soluble MR imaging agent which contains non zero nuclear spin (I≠0) nuclei which is higher than the equilibrium population difference, i.e. into a nuclear spin state polarization of the MR imaging agent.

Thus viewed from one aspect the present invention provides a method of magnetic resonance investigation of a sample, preferably a human or non-human animal body (e.g. a mammalian, reptilian or avian body), said method comprising:

(i) nuclear spin polarising a solid MR imaging agent (i.e. a material containing in its molecular structure a non-zero nuclear spin nucleus, preferably a high T₁ agent, especially preferably a water-soluble high T₁ agent) by

(a) spin refrigeration, or by

(b) irradiating with circularly polarised light;

(ii) administering the nuclear spin polarised MR imaging agent to said sample, preferably after dissolution in a physiologically tolerable solvent and also preferably after separation from some or all of the paramagnetic species or chromophores;

(iii) exposing said sample to a radiation at a frequency selected to excite nuclear spin transitions in selected nuclei therein, e.g. the spin polarised nuclei of the MR imaging agent;

(iv) detecting magnetic resonance signals from said sample; and

(v) optionally generating an image, dynamic flow data, diffusion data, perfusion data, physiological data (e.g. pH, pO₂, pCO₂, temperature or ionic concentrations) or metabolic data from said detected signals.

Thus the invention may involve the sequential steps of nuclear spin polarising (otherwise referred to herein as “hyperpolarising”) a solid MR imaging agent by polarisation transfer from paramagnetic electron spins with large anisotropy factors producing a hyperpolarised solution from said high T₁ agent, administering the hyperpolarised MR imaging agent (preferably in solution but optionally as a finely divided particulate, and preferably in the absence of a portion of, more preferably substantially the whole of, the paramagnetic species involved in transferring the polarisation), and conventional in vivo MR signal generation and measurement. The MR signals obtained in this way may be conveniently converted by conventional manipulations into 2-, 3- or 4-dimensional data including flow, diffusion, physiological or metabolic data.

Simply placing the MR imaging agent and a paramagnetic species under the low temperature and high field environment of spin refrigeration will cause a greater nuclear spin polarisation in the MR imaging agent than the equilibrium polarisation at ambient temperature and magnetic field. This polarisation is increased still further by the spin refrigeration achieving a polarisation preferably in excess of 0.1%, more preferably 1%, even more preferably 10%, yet more preferably in excess of 30%.

Wherein the nuclear spin polarising of the MR imaging agent is by irradiating with circularly polarised light, steps (i) and (ii) of the method of the invention comprises the following:

i) irradiating a solid compound having a singlet electronic ground state and containing a non zero nuclear spin nucleus with light to generate an excited polarized triplet electronic state of said agent;

ii) transforming electronic polarization of said solid compound into a nuclear spin polarization in a soluble solid MR imaging agent to form a nuclear spin polarised MR imaging agent;

iii) dissolving said polarised MR imaging agent in an aqueous medium, preferably a physiologically tolerable medium, e.g. water;

iv) administering said solution to said sample;

v) exposing said sample to radiation of a frequency selected to excite nuclear spin transitions of said non-zero nuclear spin nuclei;

vi) detecting magnetic resonance signals of said non-zero nuclear spin nuclei from said sample; and

vii) optionally, generating an image or biological functional data or dynamic flow data from said detected signals.

Viewed from a further aspect the invention provides a process for the preparation of a nuclear spin polarised MR imaging agent, said process comprising irradiating a solid compound having a singlet electronic ground state and containing a non zero nuclear spin nucleus with light to generate an excited polarized triplet electronic state of said agent;

transforming electronic polarization of said solid compound into a nuclear spin polarization in a soluble solid MR imaging agent to form a nuclear spin polarised MR imaging agent, optionally dissolving said MR imaging agent in an aqueous medium (preferably a physiologically tolerable medium), and optionally storing said polarised MR imaging agent at a reduced temperature, e.g. at liquid nitrogen temperature or below, for example at 10 K (the working temperature of a commercial closed-cycle cryo-cooler (APD-cryogenics)) or liquid helium at 4.2 K, and at a magnetic field of greater than 10 mT, preferably greater than 0.1 T, more preferably greater than 0.5 T, even more preferably greater than 2 T.

The process of nuclear spin polarisation in the method of the invention involving irradiating with circularly polarised light essentially involves two stages. First, a polarised electronic triplet state must be formed and second this electronic polarisation is harnessed to generate a nuclear spin polarisation.

By a polarised electronic triplet state is meant the case where the three sub-levels of the triplet state are not equally populated.

Maximum electronic polarisation obviously occurs when only one of the three triplet sublevels is populated. There are several different ways to achieve the polarised electronic triplet states.

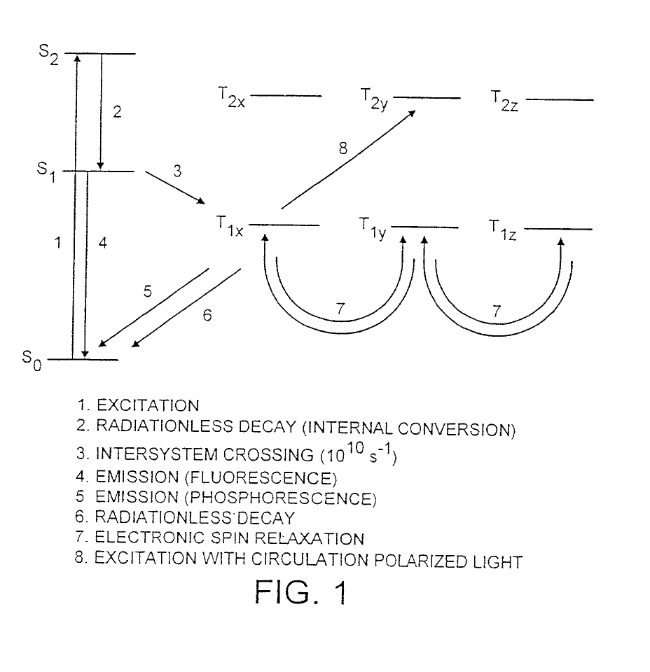

The interactions between the electronic singlet and triplet states of a photoactive molecule are shown schematically in FIG. 1 of the accompanying drawings.

The lowest electronic triplet state, T₁, is formed by intersystem crossing from the first excited singlet state, S₁, which can be reached from the singlet ground state, S₀, by light absorption and internal conversion (radiationless decay). This triplet state, T₁, has three different sub-levels, T₁ₓ, T_{1y} and T_{1z}, which are populated to different extents by the intersystem crossing. This has the advantage that a low temperature is not required for the generation of the polarised electronic triplet state. It has, however, the disadvantage of relatively poor efficacy where the irradiated solid MR imaging agent is a powder where many different crystals with different orientations are mixed. To use this effect it is desirable that the lifetime of the triplet state is short compared to the lifetime of the individual triplet sublevels (i.e. the rate of decay of the triplet state should be faster than the rate of interconversion between different triplet sub-levels). This is quite common but it is even more common that the opposite is true in the solid state, especially at lower temperatures.

It is also possible to utilise the opposite situation when the lifetime of the triplet state is long compared to the triplet sub-level lifetime. Polarisation of the electrons then depends only on the temperature. This has to be in the one Kelvin range to polarise the electrons efficiently, since it relies on the triplet sub-levels not being absolutely degenerate.

A third way of generating polarised electronic triplets has not been used before for solid materials. If the triplet state is irradiated with positively, circularly polarised light of such a wavelength that it is in resonance with the T₁-T₂ transition, where T₂ is the next highest electronic triplet state above T₁, only transitions where the magnetic and the electronic quantum number are both increased are allowed. This means that for a hypothetical case, T₁ₓ-T_{2y}, T_{1y}-T_{2z} transitions are allowed but no transitions from T_{1z} will be allowed. The T_{2z} state then relaxes quickly to give a mixture of the three sublevels of T₁. Thus the process provides a net transfer of population from T₁ₓ and T_{1y} to T_{1z}. After a number of such cycles, T₁ₓ and T_{1y} will have been depleted and T_{1z} will hold a large population. This technique is especially attractive since there are no demands on temperature or on the presence of a strong magnetic field for the generation of the polarised electronic triplets, and indeed this technique forms a further aspect of the present invention.

Thus viewed from a further aspect, the present invention provides a process for the preparation of a polarised electronic triplet state of a solid compound having a singlet electronic ground state, preferably a water-soluble compound containing at least one non-zero nuclear spin nucleus, said process comprising irradiating said compound in a solid state with a first radiation (i.e. light) of a wavelength selected to excite said compound from a ground singlet electronic state to an excited singlet electronic state and with a positively or negatively, circularly polarised second radiation of a wavelength selected to excite said compound from the lowest triplet electronic state to the next-to-lowest triplet electronic state.

The second part of the nuclear spin polarisation process involves an efficient transfer of polarisation from the electrons to non zero nuclear spin nuclei in the solid material. The I≠0 nuclei in question may be in the electronically polarised compound or may be in a separate compound mixed therewith. Preferably, however, the MR imaging agent is the same as the compound which is excited into a polarised triplet electronic state.

Due to the relatively large difference in energy between the electron spin transitions and the nuclear spin transitions, spontaneous polarisation transfer is rather slow. However, this can be remedied by Hartman-Hahn matching where the energy difference is supplied by an external radio source. This is a pulsed technique that is quite demanding when it comes to field homogeneity, transmitter power, and radio electronics and is described by Henesta et al. in J. Magn. Resonance 77: 389 (1988). Technically simpler to use is the solid effect where forbidden transitions involving both electron and nuclear spin flips are excited. There is also an improved version called the integrated solid effect which is described by van den Heuvel et al. in Chem Phys 187: 365 (1994). The acronym MIONP (Microwave Induced Optical Nuclear Polarisation) has been used for the combination of optically generated triplets with microwave irradiation for polarisation transfer. These two effects and a similar technique called thermal mixing are described together in more detail below.

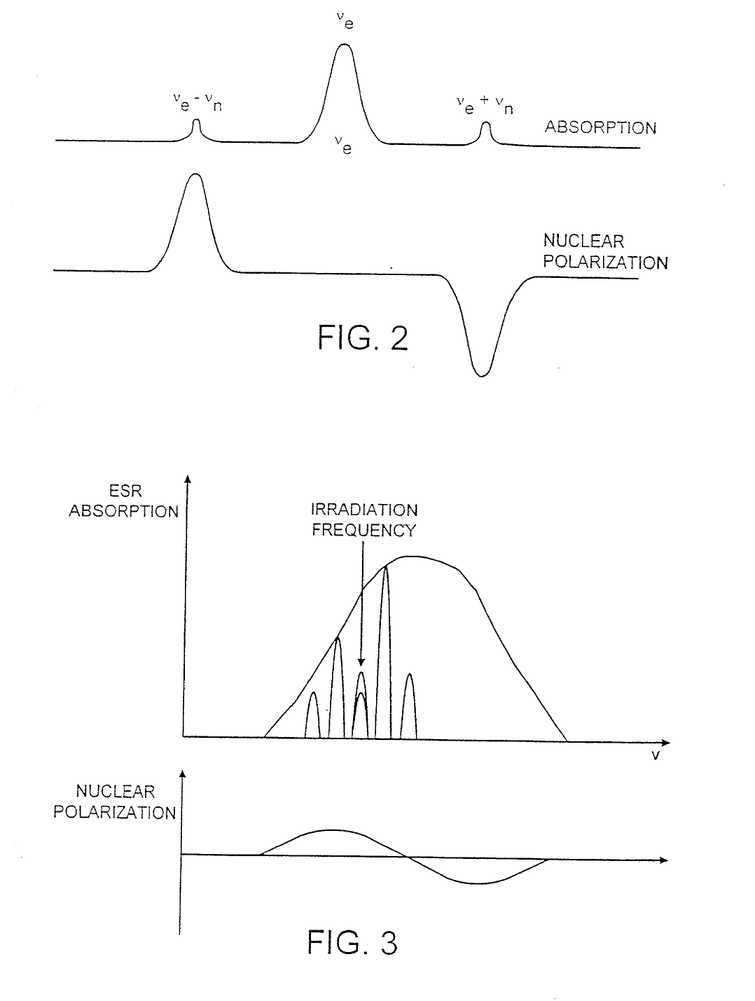

The solid effect in its pure form occurs in a material that has been doped with a paramagnetic species that has an ESR linewidth, Δνₑ, that is smaller than the resonance frequency of the nuclear spin νₙ at a given magnetic field, as shown in FIG. 2 of the accompanying drawings. The solid effect works at two frequencies, νₑ−νₙ and νₑ+νₙ. The transitions involves the simultaneous inversion of an electron and a nuclear spin, a process which is forbidden to a first approximation and slow in real time.

In FIG. 2 is shown the appearance of a hypothetical, ideal absorption mode ESR spectrum with narrow lines and the two forbidden transitions. Below it is shown the nuclear polarisation as a function of the ESR excitation frequency.

The solid effect gradually changes to what is called the differential solid effect as the linewidth of the unpaired electron becomes equal to or greater than the resonance frequency of the nuclear spin. This means that at low fields the differential solid effect will be the normal case.

The ESR line of a solid material will generally be inhomogenously broadened, that is, it can be looked upon as a collection of spin packets with slightly different resonance frequencies. As can be seen in FIG. 3 of the accompanying drawings, it is impossible to cleanly irradiate one of the forbidden transitions that lead to nuclear polarisation. Instead there will always be a mixture of irradiation of transitions that lead to positive polarisation as well as those leading to negative polarisation. The net effect will only be the difference between the rates of positive and negative polarisation, hence the term differential solid effect.

As mentioned above, the differential solid effect will lead to poor efficiency at low magnetic fields or with broad ESR lines. This can be remedied by use of the integrated solid effect, in which the irradiation frequency is swept from one side of the line to the other. Assuming the direction of the sweep is from low to high frequency, the effect for one spin packet will then be that initially the forbidden transition leading to positive polarisation will be encountered and utilised, leading to a build-up of the nuclear polarisation. As the frequency increases, the main ESR absorption of the electron will be irradiated and the population is inverted. Now, when the high frequency forbidden transition is irradiated it will also lead to positive polarisation of the nuclear spins since the electron population has been inverted. There are certain conditions for the sweep time and irradiation intensity for this effect to work well and these can be found in the 1997 thesis of M. Iinuma, University of Kyoto, entitled “Dynamic nuclear polarisation at high temperature for polarised proton target”, the contents of which are incorporated herein by reference. This is the preferred mode of operation for efficient polarisation transfer according to the present invention.

When the concentration of unpaired spins is high enough, a process called thermal mixing may be utilised. As opposed to the solid effect described above, this is an allowed process. The requirement is that the linewidth of the ESR absorption is larger than the nuclear Larmor frequency. To understand what happens in thermal mixing, assume that a microwave photon is absorbed at the high-energy side of the line. The excited electron now has the correct energy to flip-flop with an electron spin at the low energy end of the microwave line and a nuclear spin at the same time. This will transfer polarisation from electrons to nuclei.

The methods previous described in the art all rely upon the doping of proton rich materials with molecules with good photophysical characteristics. However, this is not an altogether satisfactory approach to producing hyperpolarised contrast agents since the dopant has either to be non-toxic or to be effectively removed before injection. More convenient would be to incorporate good photophysical characteristics, a long T₁ relaxation time (both in solid form and in solution), good water solubility, and low toxicity in a single molecule. Generally, this would require a molecule with a ¹³C nuclei, or other nuclei with long T₁ (e.g. ¹⁹F, ³Li, ¹H, ¹⁵N, ²⁹Si or ³¹P nuclei), preferably ¹³C or ¹⁵N nuclei, most preferably ¹³C nuclei. Hydrophilic groups are also desirably present in the agents, both to improve water solubility and to lower toxicity, whilst at the same time not increasing the correlation time of motion in solutions.

The non zero nuclear spin nucleus in the MR imaging agent may be present in its naturally occurring isotopic abundance. However where the nucleus is a non-preponderant isotope (e.g. ¹³C where ¹²C is the preponderant isotope) it will generally be preferred that the nucleus is present at a higher than normal level.

The presence of a chromophore in the agent is desirable if light absorption is desired and suitable examples include carbonyl groups, auxochromes, e.g. chlorine or bromine atoms, which enhance extinction coefficients of chromophores they are attached to, are also preferably present. These substituents both enhance the extinction coefficient and the efficiency of the intersystem crossing. Heterocyclic chromophores are also quite attractive since they often have high intersystem crossing efficiency, good water solubility, and are easy to label with ¹³C.

Thus viewed from a further aspect the present invention provides the use of a water-soluble, heterocyclic chromophore-containing compound containing an I=½ nucleus (preferably ¹³C or ¹⁵N) for the manufacture of an MR imaging composition for use in a method of diagnosis involving generation of an MR image by MR imaging of a human or non-human animal body, said manufacture comprising nuclear spin polarisation of said compound in the solid state and dissolution of the nuclear spin polarised compound in an aqueous medium.

Spin refrigeration requires that the MR imaging agent be doped with or be intimately mixed with (e.g. milled together with) a paramagnetic material, e.g. paramagnetic metal ions. The paramagnetic material preferably has a Landé g-tensor where one of the principal components is less than or equal to 0.004 and where the other principal component is at least 0.01, preferably at least 0.1, more preferably at least 1, or even more prferably at least 10. Examples of suitable paramagnetic species include transition metal ions, for example Ni²⁺ ions, lanthanide and actinide ions, especially lanthanide ions, in particular Ce³⁺ and Yb³⁺, most especially Ce³⁺ and Yb³⁺ ions in crystals with a symmetry axis of order three or more.

Such paramagnetic ions will reduce the relaxation times of the imaging nuclei in the MR imaging agent and thus they are preferably separated as thoroughly as possible from the MR imaging agent once spin refrigeration has taken place. Preferably at least 80% of the paramagnetic material is removed, particularly preferably 90% or more, especially preferably 95% or more, most especially 99% or more. In general, it is desirable to remove as much as possible prior to administration to improve physiological tolerability and to increase T₁. Thus preferred polarisation transfer agents (the paramagnetic substances) for use in the method according to the invention are those which can be conveniently and rapidly separated from the polarised MR imaging agent using known techniques as discussed below. However where the polarisation transfer agent is non-toxic, the separation step may be omitted.

In the separation step of the method of the invention, it is desirable to remove substantially the whole of the polarisation transfer agent from the composition (or at least to reduce it to physiologically tolerable levels) as rapidly as possible. Many physical and chemical separation or extraction techniques are known in the art and may be employed to effect rapid and efficient separation of the polarisation transfer agent and high T₁ agent. Clearly the more preferred separation techniques are those which can be effected rapidly and particularly those which allow separation in less than one second. Separation can be achieved for example by dissolving the spin polarised MR imaging agent in a solvent (or solvent mixture) and passing the resultant solution through a cation exchange medium or another cation immobilising system (e.g. a cation exchange resin or an immobilised chelating agent) or by filtering the solution where a paramagnetic material which is not soluble in the solvent system has been used or by precipitation of the paramagnetic metal from solution followed by filtration. Dissolution in a physiologically tolerable solvent, followed by passage through a cation exchange resin is preferred as it is rapid and yields a solution which can be used without further treatment.

By “physiologically tolerable solvent” we mean any solvent, solvent mixture or solution that is tolerated by the human or non-human animal body, e.g. water, aqueous solutions such as saline or aqueous alkanolic solutions, perfluorocarbons, etc.

In the “spin refrigerator” technique, where the MR imaging agent is in the form of a paramagnetic ion doped crystal, the doped crystal is cooled, e.g. to lower than 80 K, more preferably lower than 20 K, even more preferably lower than 4.2 K, most preferably lower than or equal to 1 K. This may be done by immersion in a liquid helium bath, preferably pumped to 1 K. The crystal is mounted in such a way that it can be rotated, thus enabling the axis of symmetry of the crystal field to make any angle with the main magnetic field. The magnetic field is preferably greater than 10 mT, more preferably greater than 0.1 T, even more preferably greater than 0.5 T, yet more preferably greater than or equal to 1 T, e.g. 1-7 T. Should the axis of symmetry of the crystal be threefold, or even higher, then the system is uniaxial with respect to the second-rank g-tensor, i.e. there are only two distinct principal components,

g∥=g_{zz}

and

g⊥=gₓₓ=g_{yy}.

Preferably, one of the two principal components, either g∥ or g⊥, should be at least as small as the g-factor of the nucleus, whilst the other, either g⊥ or g∥, should be much larger. In such cases, the orientation dependence of the g-factor can be written as:

g=(g∥² cos²θ+g⊥² sin²θ)^{½}

where θ=angle between the crystal symmetry axis and the magnetic field.

In addition to an anisotropic g-factor, preferably the spin lattice relaxation time of the ion is anisotropic, i.e. the relaxation time should preferably depend on the orientation of the crystal with respect to the magnetic field. Preferably, an orientation producing a large g-factor should coincide with a short relaxation time, whilst one corresponding to a g-factor equal to the nuclear g-factor should have a long relaxation time.

In the preferable case when the crystal is oriented such that the relaxation time is short and the g-factor is large, the ions and the nuclei are thermally separated and therefore the ions will quickly become polarised. On the other hand, rotating the crystal to an orientation corresponding to a long relaxation time and a small g-factor will reduce the spin temperature of the paramagnetic ion and the two spin systems will now be in thermal contact and at the same time isolated from the lattice. Thermal mixing will reduce the spin temperature of the nuclei and increase the temperature of the ions. Rotation back to the original orientation will cool the ions to their initial temperature, i.e. the lattice temperature. Preferably, the whole procedure is cyclically repeated to achieve maximum polarisation.

The technique of spin refrigeration is not limited to the polarisation of single crystals, although this is the preferred case. Nevertheless, the technique can be used for powder samples. In the latter case, the efficiency of the technique is reduced compared to the polarisation of single crystals, with each crystallite in the powder developing its own polarisation. With powder samples, the average polarisation will be 87% (10 π/36) of the polarisation of an optimally oriented single crystal.

As described above, in the spin refrigeration technique, the crystal can be rotated physically. As an alternative to physical rotation, the magnetic field can be rotated electronically, the advantage being that this enables both discrete and rapid rotation which is more efficient than continuous rotation of the crystal.

In the standard spin refrigeration method, the large magnetic anisotropi within an ion in a crystal is utilised. In order to achieve efficient thermal contact between the cooled ion and the nuclei, it is essential that the energy difference between the Zeeman levels in the cooled situation is nearly the same as for the nuclei.

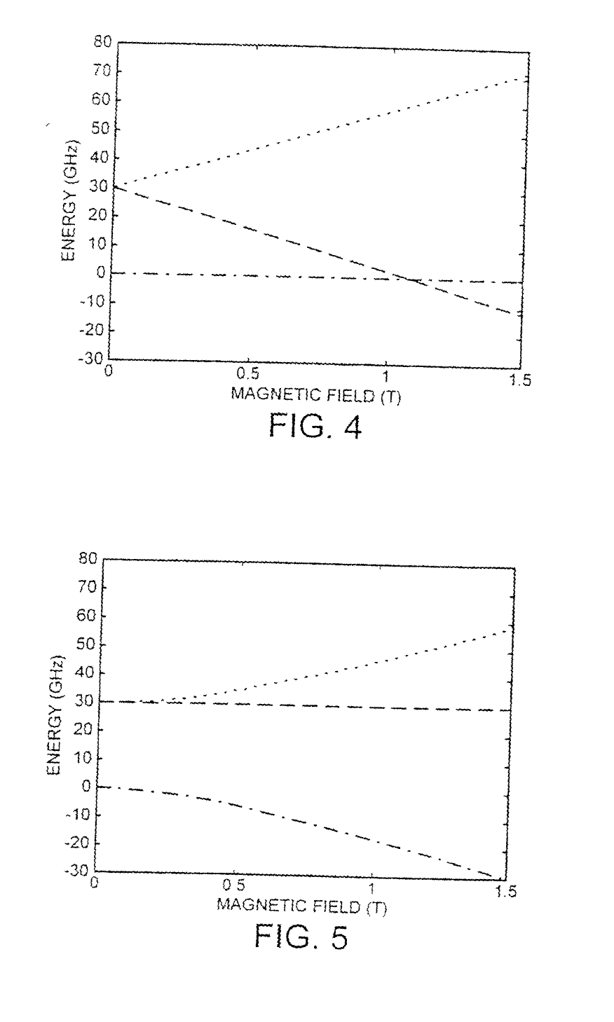

The required energy correspondence between the electronic and nuclear transitions is also found for many ions when the c-axis is parallel to the magnetic field when crossing of the lowest electronic Zeeman levels may occur. An example of such an ion is Ni²⁺ in sapphire. The nickel ion has a spin of S=1, corresponding to three Zeeman levels, and a nearly isotropic g-factor of 2. The lowest level is a singlet and in zero field a Kramers doublet is found at 30 GHz above the singlet (see FIG. 4 of the accompanying drawings). As the field increases, the lowest of the doublet levels will approach the singlet and at a field of about 1 T the two levels will cross each other, and hence the electronic and nuclear Zeeman transition energies will be the same.

If the c-axis is now turned perpendicular to the field direction, ΔE at the same field valve changes to more than the zero field splitting of 30 GHz, thus producing a large population difference between the levels (see FIG. 5 of the accompanying drawings).

In the case considered, it is assumed that the c-axis is exactly perpendicular to the axis of rotation. If, however, the c-axis is tilted slightly away from this position, ΔE will have a higher minimum valve than zero. By proper adjusting of the tilting, it is possible to optimise ΔEₘᵢₙ to the actual energy levels of the nuclei. This adjustment will extend the time the system will spend with the optimum ΔE and thus forms a further aspect of the present invention.

The spin refrigeration technique described has several major advantages over conventional dynamic nuclear polarisation (DNP) techniques. First, the instrumentation required in the spin refrigeration technique is much simpler than that required for DNP. Specifically, there is no need for a uniform magnetic field and thus no complex electronics are required. Second, crystalline powders can be used in this technique.

Specific examples of systems in which spin refrigeration has been successfully used to transfer polarisation include:

(1) Ce₂Mg₃(NO₃)₁₂.24H₂O, and

(2) Y(C₂H₅SO₄)₃.9H₂O, yttrium ethyl sulphate (YES), doped with Yb³⁺ ions. (In this system, YES has been shown to achieve proton polarisations exceeding 80%).

One embodiment of the invention provides a method as described above wherein the hyperpolarised solid sample of the MR imaging agent retains its polarisation when transported in a substantially uniform magnetic field and at low temperature; in this way the agent can be hyperpolarised at a site remote from its end use and transported to its place of use in a magnetic field and at a low temperature and there dissolved and administered.

In the embodiment referred to above, the magnetic field is preferably greater than 10 mT, more preferably greater than 0.1 T, even more preferably greater than 0.5 T, yet more preferably greater than 1 T. Alternatively it can be transported in a low temperature transporter as described in WO 99/17304. By “low temperature” in this context is preferably meant lower than 80 K, more preferably lower than 4.2 K, most preferably about 1 K.

A further embodiment of the invention provides a method as described above wherein the hyperpolarised solution thus formed retains its polarisation when transported in a magnetic field, and preferably at a low temperature, i.e. in frozen form. In this embodiment, the magnetic field is preferably greater than 10 mT, more preferably greater than 0.1 T, even more preferably greater than 0.5 T, yet more preferably greater than 1 T.

A yet further embodiment of the invention provides a method as described above wherein a magnetic field is present during the dissolution stage. In this latest embodiment, the magnetic field is preferably greater than 10 mT, more preferably greater than 0.1 T, even more preferably greater than 0.5 T, yet more preferably greater than 1 T. Examples of compounds which may be used as MR imaging agents according to the method of the invention involving irradiating with circularly polarised light include

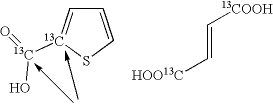

One of these positions should be labeled

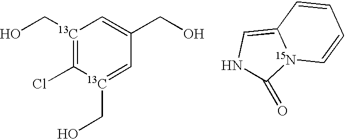

where one or more ring carbons are optionally replaced by ¹³C, carboxy groups are optionally replaced by hydroxyalkyloxycarbonyl or hydroxyalkylaminocarbonyl groups, non-labile hydrogens are optionally replaced by ²H and ring carbons in heterocyclic rings are optionally substituted by solubilising groups, e.g. hydroxyalkyl, hydroxyalkylaminocarbonyl, hydroxyalkylcarbonylamino groups, and where alkyl groups unless otherwise stated conveniently contain up to six carbons. For in vivo imaging, the MR imaging agent should of course be physiologically tolerable or be capable of being presented in a physiologically tolerable form.

The MR imaging agent should preferably be strongly nuclear spin polarisable (for example, to a level of greater than 5%, preferably greater than 10%, more preferably greater than 25%) and have an MR imaging nucleus with a long T₁ relaxation time under physiological conditions, e.g. ¹³C, ¹⁵N or ²⁹Si. By a long T₁ relaxation time is meant that T₁ is such that once nuclear spin polarised, the MR imaging agent will remain so for a period sufficiently long to allow the imaging procedure to be carried out in a comfortable time span. Significant polarisation should therefore be retained for at least 1 s, preferably for at least 60 s, more preferably for at least 100 s and especially preferably 500 s or longer.

Quadrupolar nuclei (e.g. ¹⁴N), should preferably not be included in the MR imaging agent although they may be present in counterions or other dissolved components of a contrast medium containing the MR imaging agent.

The MR imaging agent should preferably be relatively small (e.g. molecular weight less than 500 D, more preferably less than 300 D (e.g. 50-300 D) and more preferably 100 to 200 D) and also preferably should be soluble in a liquid solvent or solvent mixture, most preferably in water or another physiologically tolerable solvent or solvent mixture. Furthermore, the chemical shift, or even better the coupling constant of the nmr signal from the imaging nucleus in the MR imaging agent should preferably be influenced by physiological parameters (e.g. morphology, pH, metabolism, temperature, oxygen tension, calcium concentration, etc). For example, influence by pH can be used as a general disease marker, whilst influence by metabolism may be a cancer marker. Alternatively, the MR imaging agent may conveniently be a material which is transformed (e.g. at a rate such that its half life is no more than 10×T₁ of the reporter nucleus, preferably no more than 1×T₁) in the subject under study to a material in which the MR imaging nucleus has a different coupling constant or chemical shift. In this case the subject may be inanimate or animate, e.g. a human or animal, a cell culture, a membrane-free culture, a chemical reaction medium, etc. Thus for example the reporter nucleus may provide information on the operation of the biochemical machinery of an organism where that machinery transforms the MR imaging agent and in so doing changes the chemical shift or coupling constant of the reporter nucleus. It will be appreciated that the imaging process used in this case may be an nmr spectroscopic procedure rather than (or in addition to) an imaging procedure which generates a morphological image.

The MR imaging agent should preferably be ¹³C or ¹⁵N enriched, particularly preferably ¹³C enriched. Preferred MR imaging agents according to this aspect of the invention also exhibit the property of low toxicity.

Viewed from a further aspect the invention provides a water-soluble MR imaging agent compound:

(i) containing a nuclear spin polarised I=½ nucleus;

(ii) having a molecular weight preferably below 1000 D, more preferably below 500 D;

(iii) containing a cyclic, preferably heterocyclic, chromophore; and

(iv) having an nmr spectrum for said I=½ nucleus having a linewidth of less than 100 Hz, preferably below 10 Hz, more preferably below 1 Hz.

The MR imaging agent compound of the invention preferably contains as said I=½ nucleus a nucleus such as ¹H, ¹³C, ¹⁵N or ²⁹Si, especially ¹³C. Preferably it also has some or all of the desired properties discussed earlier, e.g. solubility, paucity of other I 0 nuclei (although these may be present in a counterion component of the compound if it is ionic), solubility in water, etc.

While compounds meeting these criteria can be used according to the invention without enrichment in ¹³C, ¹⁵N or ²⁹Si, it is preferred that they be enriched.

Suitable MR imaging agents, e.g. high T₁ agents, for use in the method of the invention involving spin refrigeration may contain nuclei such as protons. However other non-zero nuclear spin nuclei may be useful (e.g. ¹⁹F, ³Li, ¹³C, ¹⁵N, ²⁹Si or ³¹P, as well as ¹H), preferably ¹H, ¹³C ¹⁵N ¹⁹F ²⁹Si and ³¹P nuclei, with ¹³C and ¹⁵N nuclei being particularly preferred. In this event the MR signals from which the image is generated may be substantially only from the MR imaging agent itself. Nonetheless, where the polarised MR imaging agent is present in high concentration in administrable media, there may be significant enough transfer of magnetisation to the protons to be able to perform ¹H-MRI on the protons of the media. Similarly, the polarised MR imaging agent may have a significant enough effect on in vivo protons for conventional ¹H-MRI to be carried out on those protons.

Where the MR imaging nucleus is other than a proton (e.g. ¹³C or ¹⁵N), there will be essentially no interference from background signals (the natural abundance of ¹³C and ¹⁵N being negligible) and image contrast will be advantageously high. This is especially true where the MR imaging agent itself is enriched above natural abundance in the MR imaging nucleus. Thus the method according to the invention has the benefit of being able to provide significant spatial weighting to a generated image. In effect, the administration of a polarised MR imaging agent to a selected region of a sample (e.g. by injection) means that the contrast effect may be localised to that region. The precise effect of course depends on the extent of biodistribution over the period in which the MR imaging agent remains significantly polarised. In general, specific body volumes (i.e. regions of interest such as the vascular system or specific organs such as the brain, kidney, heart or liver) into which the agent is administered may be defined with improved signal to noise (particularly improved contrast to noise) properties of the resulting images in these volumes.

In one embodiment, a “native image” of the sample (e.g. body) (i.e. one obtained prior to administration of the MR imaging agent or one obtained for the administered MR imaging agent without prior polarisation as in a conventional MR experiment) may be generated to provide structural (e.g. anatomical) information upon which the image obtained in the method according to the invention may be superimposed. A “native image” is generally not available where ¹³C or ¹⁵N is the imaging nucleus because of the low abundance of ¹³C and ¹⁵N in the body. In this case, a proton MR image may be taken to provide the anatomical information upon which the ¹³C or ¹⁵N image may be superimposed.

The MR imaging agent should of course be physiologically tolerable or be capable of being provided in a physiologically tolerable, administrable form where the sample is animate. Preferred MR imaging agents are soluble in aqueous media (e.g. water) and are of course non-toxic where the intended end use is in vivo.

Conveniently, the MR imaging agent once polarised will remain so for a period sufficiently long to allow the imaging procedure to be carried out in a comfortable time span. Generally sufficient polarisation will be retained by the MR imaging agent in its administrable form (e.g. in injection solution) if it has a T₁ value (at a field strength of 0.01-5 T and a temperature in the range 20-40° C.) of at least 5 s, more preferably at least 10 s, especially preferably 30 s or longer, more especially preferably 70 s or more, yet more especially preferably 100 s or more (for example at 37 C. in water at 1 T and a concentration of at least 1 mM). The MR imaging agent may be advantageously an agent with a long T₂ relaxation time.

The long T₁ relaxation time of certain ¹³C nuclei is particularly advantageous and certain MR imaging agents containing ¹³C nuclei are therefore preferred for use in the present method. The γ-factor of carbon is about ¼ of the—factor for hydrogen resulting in a Larmor frequency of about 10 MHz at 1 T. The rf-absorption and reflections in a patient is consequently and advantageously less than in water (proton) imaging. The signal-to-noise ratio is found to be independent of the MRI field strength when the corresponding frequency is higher than a few MHz. Preferably the polarised MR imaging agent has an effective ¹³C nuclear polarisation corresponding to the one obtained at thermal equilibrium at 300 K in a field of 0.1 T or more, more preferably 25 T or more, particularly preferably 100 T or more, especially preferably 5000 T or more (for example 50 kT). When the electron cloud of a given molecule interacts with atoms in surrounding tissue, the shielding of the atom responsible for the the MR signal is changed giving rise to a shift in the MR frequency (“the chemical shift effect”). When the molecule is metabolised, the chemical shift will be changed and MR imaging agents in different chemical surroundings may be visualised separately using pulses sensitive to chemical shift. When the frequency difference between MR imaging molecules in different surroundings is 10 Hz or higher, preferably 20 Hz or higher, most preferably 150 Hz or higher (corresponding to 3.5 ppm or higher), the two components may be excited separately and visualised in two images. Standard chemical shift selective excitation pulses may then be utilised. When the frequency separation is less, the two components may not be separated by using frequency selective rf-pulses. The phase difference created during the time delay after the excitation pulse and before the detection of the MR signal may then be used to separate the two components. Phase sensitive imaging pulse sequence methods (Dixon, Radiology, 1984, 153: 189-194 and Sepponen, Mag Res. Imaging, 3, 163-167, 1985) may be used to generate images visualising different chemcial surroundings or different metabolites. The long T₂ relaxation time which may be a characteristic of a high T₁ agent will under these circumstances make it possible to use long echo times (TE) and still get a high signal-to-noise ratio. Thus an important advantage of the MR imaging agents used in the present method is that they exhibit a chemical shift dependent on the local composition of the body in which they are localised. Preferred MR imaging agents will exhibit a chemical shift of more than 2 ppm, preferably more than 10 ppm depending on whether the MR imaging agent is localised inside or outside the vascular system. More preferred MR imaging agents will exhibit a chemical shift of more than 2 ppm, preferably more than 10 ppm, per 2 pH units or per Kelvin or upon being metabolised. MR imaging agents containing polarised ¹³C nuclei (or ¹⁵N nuclei) exhibit large changes in chemical shift in response to physiological changes (e.g. pH, pO₂, pCO₂, redox potential, temperature or ionic concentrations of for example Na⁺, K⁺, Ca²⁺) or metabolic activity and therefore may be used to monitor these parameters.

Alternatively, the T₂ valve may be sensitive to the physiological parameters of interest.

Solid MR imaging agents (e.g. ¹³C or ¹⁵N enriched solids) may exhibit very long T₁ relaxation times and for this reason are especially preferred for use in the present method. The T₁ relaxation time may be several hours in the bulk phase, although this may be reduced by reduction of grain size and/or addition of paramagnetic impurities e.g. molecular oxygen. The long relaxation time of solids advantageously allows the procedure to be conveniently carried out with less haste and is particularly advantageous in allowing the polarised solid MR imaging agent to be stored or transported prior to pharmaceutical formulation and administration. In one embodiment, the polarised MR imaging agent may be stored at low temperature and prior to administration, the MR imaging agent may be rapidly warmed to physiological temperatures using conventional techniques such as infrared or microwave radiation or simply by adding hot, sterile administrable media e.g. saline.

For in vivo use, a polarised solid MR imaging agent is dissolved in administrable media (e.g. water or saline), administered to a subject and conventional MR imaging performed. Thus solid MR imaging agents are preferably rapidly soluble (e.g. water soluble) to assist in formulating administrable media. Preferably the MR imaging agent should dissolve in a physiologically tolerable carrier (e.g. water or Ringers solution) to a concentration of at least 1 mM at a rate of 1 mM/3 T₁ or more, particularly preferably 1 mM/2 T₁ or more, especially preferably 1 mM/T₁ or more. Where the solid MR imaging agent is frozen, the adminstrable medium may be heated, preferably to an extent such that the temperature of the medium after mixing is close to 37 C.

A polarised MR imaging agent may be administered (either alone or with additional components such as additional MR imaging agents) in liquid form. The retention of polarisation in a liquid medium vis-a-vis a gas medium is significantly greater. Thus while T₁ and T₂ are in general shorter for the liquid, the T₂* effect due to diffusion is 10⁵ times less significant for the liquid. Consequently for gaseous MR imaging agents the imaging sequence used generally has to be FLASH or GRASS while in contrast, more efficient imaging sequences may be used for liquids. For example, liquids generally have slower diffusion which makes it possible to use sequences such as echo planar imaging (EPI). The overall technique will be faster and yield better resolution (voxel size <1 mm) than conventional techniques (voxel size approx. 1-5 mm) at current acquisition times. It will give good images at all fields including in low field (e.g. 0.01-0.5 T) machines.

Unless the hyperpolarised agent is stored (and/or transported) at low temperature and in an applied field as described above, since the method of the invention should be carried out within the time that the hyperpolarised solution of the MR imaging agent remains significantly polarised, it is desirable for administration of the polarised MR imaging agent to be effected rapidly and for the MR measurement to follow shortly thereafter. The preferred administration route for the polarised MR imaging agent is parenteral e.g. by bolus injection, by intravenous, intraarterial or peroral injection. The injection time should be equivalent to 5 T₁ or less, preferably 3 T₁ or less, more preferably T₁ or less, especially 0.1 T₁ or less. The lungs may be imaged by spray, e.g. by aerosol spray.

The MR imaging agent should be preferably enriched with nuclei (e.g. ¹⁵N and/or ¹³C nuclei) having a long T₁ relaxation time. Preferred are ¹³C enriched MR imaging agents having ¹³C at one particular position (or more than one particular position) in an amount in excess of the natural abundance, i.e. above about 1%. Preferably such a single carbon position will have 5% or more ¹³C, particularly preferably 10% or more, especially preferably 25% or more, more especially preferably 50% or more, even more preferably in excess of 99% (e.g. 99.9%). The ¹³C nuclei should preferably amount to >2% of all carbon atoms in the compound. The MR imaging agent is preferably ¹³C enriched at one or more carbonyl or quaternary carbon postions, given that a ¹³C nucleus in a carbonyl group or in certain quaternary carbons may have a T₁ relaxation time typically of more than 2 s, preferably more than 5 s, especially preferably more than 30 s. Preferably the ¹³C enriched compound should be deuterium labelled, especially adjacent the ¹³C nucleus.

Preferred ¹³C enriched compounds are those in which the ¹³C nucleus is surrounded by one or more non-MR active nuclei such as O, S, C or a double bond. Specifically preferred ¹³C enriched agents are ¹³CO₃²⁻ and H¹³CO₃—(sodium salt for injection and calcium or potassium salt for polarisation).

Also preferred are the following types of compound (further details can be found in WO 99/35508 and WO 96/09282 which are herein incorporated by reference):

(1) carboxyl compounds comprising 1 to 4 carboxyl groups,

(2) substituted mono and biaryl compounds,

(3) sugars,

(4) ketones,

(5) ureas,

(6) amides,

(7) amino acids,

(8) carbonates,

(9) nucleotides, and

(10) tracers.

Viewed from a still further aspect the invention provides a physiologically tolerable MR imaging composition comprising a physiologically tolerable nuclear spin polarised MR imaging agent according to the invention dissolved in water together with one or more physiologically tolerable excipients, said imaging agent containing nuclei of a I=½ isotope (e.g. ¹³C, ¹⁵N or ²⁹Si), preferably at a higher than natural abundance, characterised in that said nuclei are polarised such that their nmr signal intensity is equivalent to a signal intensity achievable in a magnetic field of at least 0.1 T, more preferably at least 25 T, particularly preferably at least 100 T, especially preferably at least 450 T, e.g. at 21 C. Preferably, the composition is sterile and is stable at a physiologically temperature (e.g. at 10-40 C.).

Viewed from a further aspect, the present invention provides the use of a paramagnetic substance for the manufacture of an MR imaging composition for use in a method of diagnosis involving generation of an MR image by MR imaging of a human or non-human animal body, wherein manufacture of said composition involves spin refrigeration nuclear spin polarisation of said MR imaging agent.

Viewed from an alternative aspect, the invention provides the use of an MR imaging agent for the manufacture of an MR imaging composition for use in a method of diagnosis involving generation of an MR image by MR imaging of a human or non-human animal body, wherein manufacture of said composition involves spin refrigeration nuclear spin polarisation of said MR imaging agent.

Viewed from a yet still further aspect, the invention provides an MR imaging composition comprising a solution of a spin refrigerator nuclear spin polarised MR imaging agent in a physiologically tolerable solvent, optionally together with one or more physiologically tolerable excipients.

Given that the method of the invention should be carried out within the time that the MR imaging agent remains significantly polarised, once nuclear spin polarisation and dissolution has occurred, it is desirable for administration of the MR imaging agent to be effected rapidly and for the MR measurement to follow shortly thereafter. This means that the sample (e.g. body or organ) should be available close to the area in which the polarisation has been carried out. If this is not possible, the material should be transported to the relevant area, preferably at low temperature.

The preferred administration route for the MR imaging agent is parenteral, e.g. by bolus injection, by intravenous or intra-arterial injection or, where the lungs are to be imaged, by spray, e.g. by aerosol spray. Oral and rectal administration may also be used.

Where the MR imaging nucleus is other than a proton (e.g. ¹³C), there will be essentially no interference from background signals (the natural abundance of ¹³C, ¹⁵N, ²⁹Si etc. being negligible) and image contrast will be advantageously high. Thus the method according to the invention has the benefit of being able to provide significant spatial weighting to a generated image. In effect, the administration of a polarised MR imaging agent to a selected region of a sample (e.g. by injection) means that the contrast effect is, in general, localised to that region. The precise effect of course depends on the extent of biodistribution over the period in which the MR imaging agent remains significantly polarised. In general, specific body volumes (i.e. regions of interest such as the vascular system) into which the MR imaging agent is administered may be defined with improved signal to noise properties of the resulting images in these volumes.

Moreover, the γ-factor of carbon is about ¼ of the γ-factor for hydrogen resulting in a Larmor frequency of about 10 MHz at 1 T. The rf-absorption in a patient is consequently and advantageously less than in ¹H imaging. A further advantage of MR imaging agents containing polarised ¹³C nuclei is the ability to utilise large changes in chemical shift in response to physiological changes, e.g. pH or temperature.

The MR imaging agent may be conveniently formulated with conventional pharmaceutical or veterinary carriers or excipients. MR imaging agent formulations manufactured or used according to this invention may contain, besides the MR imaging agent, formulation aids such as are conventional for therapeutic and diagnostic compositions in human or veterinary medicine but will be clean, sterile and free of paramagnetic, superparamagnetic, ferromagnetic or ferrimagnetic contaminants. Thus the formulation may for example include stabilizers, antioxidants, osmolality adjusting agents, solubilizing agents, emulsifiers, viscosity enhancers, buffers, etc. Preferably none of such formulation aids will be paramagnetic, superparamagnetic, ferromagnetic or ferrimagnetic. The formulation may be in forms suitable for parenteral (e.g. intravenous or intraarterial) or enteral (e.g. oral or rectal) application, for example for application directly into body cavities having external voidance ducts (such as the lungs, the gastrointestinal tract, the bladder and the uterus), or for injection or infusion into the cardiovascular system. However solutions, suspensions and dispersions in physiological tolerable carriers (e.g. water) will generally be preferred.

For use in in vivo imaging, the formulation, which preferably will be substantially isotonic, may conveniently be administered at a concentration sufficient to yield a 1 micromolar to 1 M concentration of the MR imaging agent in the imaging zone; however the precise concentration and dosage will of course depend upon a range of factors such as toxicity, the organ targeting ability of the MR imaging agent, and the administration route. The optimum concentration for the MR imaging agent represents a balance between various factors. In general, optimum concentrations would in most cases lie in the range 0.1 mM to 10 M, especially 0.2 mM to 1 M, more especially 0.5 to 500 mM. Formulations for intravenous or intraarterial administration would preferably contain the MR imaging agent in concentrations of 10 mM to 10 M, especially 50 mM to 500 mM. For bolus injection the concentration may conveniently be 0.1 mM to 10 M, preferably 0.2 mM to 10 M, more preferably 0.5 mM to 1 M, still more preferably 1.0 mM to 500 mM, yet still more preferably 10 mM to 300 mM.

Parenterally administrable forms should of course be sterile and free from physiologically unacceptable agents and from paramagnetic, superparamagnetic, ferromagnetic or ferrimagnetic contaminants, and should have low osmolality to minimize irritation or other adverse effects upon administration and thus the formulation should preferably be isotonic or slightly hypertonic. Suitable vehicles include aqueous vehicles customarily used for administering parenteral solutions such as Sodium Chloride solution, Ringer's solution, Dextrose solution, Dextrose and Sodium Chloride solution, Lactated Ringer's solution and other solutions such as are described in Remington's Pharmaceutical Sciences, 15th ed., Easton: Mack Publishing Co., pp. 1405-1412 and 1461-1487 (1975) and The National Formulary XIV, 14th ed. Washington: American Pharmaceutical Association (1975). The compositions can contain preservatives, antimicrobial agents, buffers and antioxidants conventionally used for parenteral solutions, excipients and other additives which are compatible with the MR imaging agents and which will not interfere with the manufacture, storage or use of the products.

Where the MR imaging agent is to be injected, it may be convenient to inject simultaneously at a series of administration sites such that a greater proportion of the vascular tree may be visualized before the polarisation is lost through relaxation. Intra-arterial injection is useful for preparing angiograms and intravenous injection for imaging larger arteries and the vascular tree.

The dosages of the MR imaging agent used according to the method of the present invention will vary according to the precise nature of the MR imaging agents used, of the tissue or organ of interest and of the measuring apparatus. Preferably the dosage should be kept as low as possible whilst still achieving a detectable contrast effect. Typically the dosage will be approximately 10% of LD₅₀, eg in the range 1 to 1000 mg/kg, preferably 2 to 500 mg/kg, especially 3 to 300 mg/kg.

Viewed from a yet still further aspect, the invention provides an apparatus for use in the method described herein, the apparatus comprising:

i) a chamber cooled by, e.g. liquid helium, to a temperature preferably lower than 80 K, more preferably lower than 20 K, even more preferably lower than 4.2 K, most preferably lower than or equal to 1 K, disposed in the primary magnetic field of MR apparatus, or in a separate magnetic field, of strength 0.2 T or more, preferably 0.5 to 10 T;

and wherein said chamber is:

i) adapted to receive particulate solid MR imaging agent, doped with or intimately mixed with paramagnetic polarising agent;

ii) rotates said agent about an axis non-parallel with the primary field or passes said agent through a conduit such that it rotates in that way (e.g. in a spiral or helical conduit) or mixes said agent (e.g. by means of rotating paddles) such that it rotates in that way, or (where the chamber is in a separate magnetic field) rotates the magnetic field about one or more axes;

iii) dissolves said polarised solid agent in or passes it to a mixing chamber, where it is dissolved in a physiologically tolerable solvent;

iv) passes the solution thus formed through or over an immobilised paramagnetic metal binding agent (e.g. an ion exchange resin) and/or through a filter;

v) and into the conduit for administration into a sample (e.g. a patient) situated within the primary magnetic field of the MR imager.

In the present invention, hyperpolarisation of the solid MR imaging agent is effected by increasing the polarisation of the nucleus in said agent to be observed in said MR investigation by polarisation transfer from paramagnetic electron spins with large anisotropy factors. It is envisaged that, in the method according to the invention, the level of polarisation achieved should be sufficient to allow the hyperpolarised solution of the MR imaging agent to achieve a diagnostically effective contrast enhancement in the sample to which it is subsequently administered in whatever form. In general, it is desirable to achieve a degree of polarisation which is at least a factor of 2 or more above the equilibrium value at the temperature and the magnetic field in which MRI is performed, preferably a factor of 10 or more, particularly preferably 100 or more and especially preferably 1000 or more, e.g. 50000.

The contents of all publications referred to herein are hereby incorporated by reference.

Embodiments of the invention are described further with reference to the following non-limiting Examples and the accompanying drawings, in which:

FIG. 1 is a schematic diagram showing the interactions between the electronic singlet and triplet states of a photoactive molecule;

FIG. 2 shows the solid effect in its pure form;

FIG. 3 shows the differential solid effect;

FIG. 4 shows the energy levels of Ni²⁺ in sapphire when the c-axis is parallel to the field direction; and

FIG. 5 shows the energy levels of Ni²⁺ in sapphire when the c-axis is perpendicular to the field direction.

### EXAMPLE 1

Irradiating with Circularly Polarised Light

A sample of a compound to be nuclear spin polarised is placed in a sample holder with transparent, preferably quartz, walls. In the centre of the sample holder is a material that absorbs light and prevents the passage of light past the centre of the sample. Preferably it is a rod or tube of oxidized copper or silver or other dark material with good heat conduction properties. The charged sample holder is placed in a cooling bath, containing liquid nitrogen or helium, equipped with windows to allow for the passage of two light beams converging on the sample. This cooling bath is located in a magnetic field, of strength between 0.01 to 10 Tesla depending on the relaxation characteristics of the sample. The sample is then irradiated with light from two different sources. Source one is a low power light source with a wavelength chosen to excite molecules from the Sₒ state to one of the higher S-states. The desired wavelength is selected with a monochromator or a suitable combination of filters. The light source is typically a mercury lamp. The irradiation power is chosen to give a substantial degree of hole burning in the chosen transition. Optionally this light source may operate in a pulsed fashion. Source two is a high power light source with polariser and quarter wave plate so that a circularly polarised light is obtained. The wavelength is chosen to excite molecules in the T₁ state to the T₂ state. This wavelength is typically longer than the Sₒ-Sₙ wavelength. The power should be the highest possible compatible with the cooling capacity. The sample thickness is adjusted so that this light penetrates the whole sample. To give even polarisation throughout the material the sample is rotated, typically with a frequency of 1 to 100 Hz. After an irradiation time of 5 times the nuclear T₁, a maximum polarisation has been reached and the sample is rapidly removed from the cooling bath and poured into warm (40° C.), agitated water (optionally with pharmacological additives). It is important to keep the sample within the magnetic field during this operation and until the solids have been dissolved.

In one embodiment, the solid is nuclear spin polarised in microcrystallic or amorphous powder form, optionally agitated by a gas (e.g. He) whereby to produce a “dust-in-air suspension”.

In one experiment the aqueous solution is rapidly transferred to an NMR spectrometer and a spectrum with enhanced intensity is recorded.

In a second experiment the aqueous solution is rapidly transferred to an MRI-scanner and a picture with enhanced contrast and intensity is recorded.

In a third experiment the aqueous solution is rapidly injected into a rat, which is placed in an MRI-scanner, and a picture with enhanced contrast and intensity is recorded.

### EXAMPLE 2

Spin Refrigeration

A substrate with long T₁ times, e.g. a ¹³C or ¹⁵N-labelled compound, is milled with anisotropic metal ions. The mixture formed is placed in a sample holder and immersed in liquid helium in a 1 T magnet. A vacuum is applied to the helium bath and the sample holder is spun at 100 Hz around the axis of which is perpendicular to the external magnetic field. After several minutes the vacuum is released and the sample is rapidly removed and poured into water at 40 C. The solution thus formed is rapidly passed through an ion-exchange column to remove the anisotropic metal ions and is then ready for injection, optionally with the addition of pharmacological additives.

1. A method of magnetic resonance investigation of a sample, preferably of a human or non-human animal body, said method comprising:
(i) nuclear spin polarising a solid MR imaging agent (i.e. a material containing in its molecular structure a non-zero nuclear spin nucleus) by (a) spin refrigeration, or by, (b) irradiating with circularly polarised light;
(ii) administering the nuclear spin polarised MR imaging agent to said sample, preferably after dissolution in a physiologically tolerable solvent and also preferably after separation from some or all of the paramagnetic species or chromophores;
(iii) exposing said sample to a radiation at a frequency selected to excite nuclear spin transitions in selected nuclei therein, e.g. the spin polarised nuclei of the MR imaging agent;
(iv) detecting magnetic resonance signals from said sample; and
(v) optionally generating an image, dynamic flow data, diffusion data, perfusion data, physiological data (e.g. pH, pO₂, pCO₂, temperature or ionic concentrations) or metabolic data from said detected signals.

2. A method as claimed in claim 1 wherein said agent is administered to said sample after dissolution in water.

3. A method as claimed in either one of claims 1 and 2 wherein said agent further comprises other pharmaceutical additives.

4. A method as claimed in any one of the preceding claims wherein said solid MR imaging agent is a water-soluble high T₁ agent.

5. A method as claimed in any one of the preceding claims wherein said MR imaging agent retains its polarisation when transported in a substantially uniform magnetic field and at low temperature.

6. A method as claimed in claim 5 wherein said magnetic field is greater than 10 mT.

7. A method as claimed in claim 5 wherein said magnetic field is greater than 1 T.

8. A method as claimed in any one of claims 5 to 7 wherein said temperature is lower than 80 K.

9. A method as claimed in any one of claims 5 to 7 wherein said temperature is lower than 4.2 K.

10. A method as claimed in any one of the preceding claims wherein the solution formed retains its polarisation in frozen form.

11. A method as claimed in any one of the preceding claims wherein a magnetic field is present during the dissolution stage.

12. A method as claimed in any one of the preceding claims wherein step (i) comprises:
i) irradiating a solid compound having a singlet electronic ground state and containing a non zero nuclear spin nucleus with light to generate an excited polarized triplet electronic state of said agent;
ii) transforming electronic polarization of said solid compound into a nuclear spin polarization in a soluble solid MR imaging agent to form a nuclear spin polarised MR imaging agent;
iii) dissolving said polarised MR imaging agent in an aqueous medium.

13. Use of a paramagnetic substance for the manufacture of an MR imaging composition for use in a method of diagnosis involving generation of an MR image by MR imaging of a human or non-human animal body, wherein manufacture of said composition involves spin refrigeration nuclear spin polarisation of said MR imaging agent.

14. Use of an MR imaging agent for the manufacture of an MR imaging composition for use in a method of diagnosis involving generation of an MR image by MR imaging of a human or non-human animal body, wherein manufacture of said composition involves spin refrigeration nuclear spin polarisation of said MR imaging agent.

15. An MR imaging composition comprising a solution of a spin refrigerator nuclear spin polarised MR imaging agent in a physiologically tolerable solvent, optionally together with one or more physiologically tolerable excipients.

16. An apparatus for use in the method as claimed in claim 1 when the polarising of a MR imaging agent is by spin refrigeration, the apparatus comprising:
i) a chamber cooled to a temperature preferably lower than 80 K disposed in the primary magnetic field of MR apparatus, or in a separate magnetic field, of strength 0.2 T or more;
and wherein said chamber is:
i) adapted to receive particulate solid MR imaging agent, doped with or intimately mixed with paramagnetic polarising agent;
ii) rotates said agent about an axis non-parallel with the primary field or passes said agent through a conduit such that it rotates in that way or mixes said agent such that it rotates in that way, or (where the chamber is in a separate magnetic field) rotates the magnetic field about one or more axes;
iii) dissolves said polarised solid agent in or passes it to a mixing chamber, where it is dissolved in a physiologically tolerable solvent;
iv) passes the solution thus formed through or over an immobilised paramagnetic metal binding agent and/or through a filter;
v) and into the conduit for administration into a sample situated within the primary magnetic field of the MR imager.

17. An apparatus as claimed in claim 16 wherein said chamber is cooled to lower than or equal to 1 K.

18. An apparatus as claimed in either one of claims 16 and 17 wherein the strength of said magnetic field is 0.5 to 10 T.

19. A process for the preparation of a nuclear spin polarised MR imaging agent, said process comprising irradiating a solid compound having a singlet electronic ground state and containing a non zero nuclear spin nucleus with light to generate an excited polarized triplet electronic state of said agent;
transforming electronic polarization of said solid compound into a nuclear spin polarization in a soluble solid MR imaging agent to form a nuclear spin polarised MR imaging agent, optionally dissolving said MR imaging agent in an aqueous medium (preferably a physiologically tolerable medium), and optionally storing said polarised MR imaging agent at a reduced temperature and at a magnetic field of greater than 10 mT.

20. A process as claimed in claim 19 wherein said reduced temperature is liquid nitrogen temperature or below.

21. A process as claimed in claim 19 wherein said reduced temperature is liquid helium temperature.

22. A process as claimed in any one of claims 19 to 21 wherein said magnetic field is greater than 2 T.

23. A process for the preparation of a polarised electronic triplet state of a solid compound having a singlet electronic ground state said process comprising irradiating said compound in a solid state with a first radiation of a wavelength selected to excite said compound from a ground singlet electronic state to an excited singlet electronic state and with a positively or negatively, circularly polarised second radiation of a wavelength selected to excite said compound from the lowest triplet electronic state to the next-to-lowest triplet electronic state.

24. A process as claimed in claim 23 wherein said compound is a water-soluble compound containing at least one non-zero nuclear spin nucleus.

25. Use of a water-soluble, heterocyclic chromophore-containing compound containing an I=½ nucleus for the manufacture of an MR imaging composition for use in a method of diagnosis involving generation of an MR image by MR imaging of a human or non-human animal body, said manufacture comprising nuclear spin polarisation of said compound in the solid state and dissolution of the nuclear spin polarised compound in an aqueous medium.

26. Use as claimed in claim 25 wherein said I=½ nucleus is ¹³C or ¹⁵N.

27. A water-soluble MR imaging agent compound:
(i) containing a nuclear spin polarised I=½ nucleus;
(ii) having a molecular weight below 1000 D;
(iii) containing a cyclic chromophore; and
(iv) having an nmr spectrum for said I=½ nucleus having a linewidth of less than 100 Hz.

28. An agent compound as claimed in claim 27 wherein said molecular weight is below 500 D.

29. An agent as claimed in either one of claims 27 and 28 wherein said cyclic chromophore is heterocyclic.

30. An agent as claimed in any one of claims 27 to 29 wherein said linewidth is below 1 Hz.

31. A physiologically tolerable MR imaging composition comprising a physiologically tolerable nuclear spin polarised MR imaging agent as claimed in any one of claims 27 to 30 dissolved in water together with one or more physiologically tolerable excipients, said imaging agent containing nuclei of a I=½ isotope characterised in that said nuclei are polarised such that their nmr signal intensity is equivalent to a signal intensity achievable in a magnetic field of at least 0.1 T.

32. A composition as claimed in claim 31 wherein said nuclei are at higher than natural abundance.

33. A composition as claimed in either one of claims 31 and 32 wherein said magnetic field is at least 450 T.

34. A composition as claimed in any one of claims 31 to 33 wherein said composition is sterile and is stable at a pyysiological temperature.
